SEQUENCE LISTING (6 sequences)

SEQ ID NO: 1 - protein, 157 aa, synthetic construct
  REGION 1..157: note = Amino acid sequence of human DR-18
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 2 - protein, 157 aa, synthetic construct
  REGION 1..157: note = Amino acid sequence of murine DR-18
HFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 3 - protein, 157 aa, Homo sapiens
  REGION 1..157: note = Amino acid sequence of wild-type human IL-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 4 - protein, 157 aa, Mus musculus
  REGION 1..157: note = Amino acid sequence of wild-type murine IL-18
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 5 - DNA, 471 bp, synthetic construct
  source 1..471: mol_type = other DNA
  misc_feature 1..471: note = Nucleotide sequence of human DR-18
tatttcggcaagctggaaagcaagctctccgtgatcagaaacctgaatgatcaggtgctgttcatcgaccagggcaaccggcctctgtttgaggacatgaccgacagcgattgcagagataatgcccctagaaccatctttatcatttctaagtacagcgacagcctggctagaggcctggccgtgacaatcagcgtgaagtgcgagaagatctccaccctgagctgcgagaacaagatcatctctttcaaggaaatgaacccccccgacaacatcaaggacacaaagtccgacatcatcttcttccagagagacgtgcctggccacagcaggaagatgcagttcgagagcagcagctacgagggctacttcctggcttgtgaaaaagaacgggacctgtttaaactgatcctgaaaaaggaagatgagctgggagatagaagcatcatgttcaccgtgcagaacgaggac

SEQ ID NO: 6 - DNA, 471 bp, synthetic construct
  source 1..471: mol_type = other DNA
  misc_feature 1..471: note = Nucleotide sequence of murine DR-18
cactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacgcctacggcgacagtagagccagaggaaaggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagt

(19) United States
(12) Patent Application Publication
(10) Pub. No.: US 2026/0151445 A1
(43) Pub. Date: Jun. 4, 2026

(54) USE OF DR-18 AND ONCOLYTIC VACCINIA VIRUS IN PREPARATION OF ANTI-TUMOR DRUG

(71) Applicant: GUANGZHOU VIROTECH PHARMACEUTICAL CO., LTD., Guangdong (CN)

(72) Inventors: Ziqing LIN, Guangdong (CN); Guang'en WU, Guangdong (CN); Songmin HE, Guangdong (CN); Yichun LIU, Guangdong (CN); Shoufang GONG, Guangdong (CN)

(21) Appl. No.: 19/133,073

(22) PCT Filed: Dec. 7, 2023
(86) PCT No.: PCT/CN2023/137125

§ 371 Date: May 27, 2025

(30) Foreign Application Priority Data
Dec. 7, 2022 (CN) 202211566283.7

(51) Int. Cl. A61K 35/768; A61K 38/20; A61P 35/00; C12N 7/00

(52) U.S. Cl. CPC A61K 35/768; A61K 38/20; A61P 35/00; C12N 7/00; C12N 2710/24122; C12N 2710/24132; C12N 2710/24171

(57) ABSTRACT

The present disclosure belongs to the field of biological medicines, and relates to use of DR-18 and an oncolytic vaccinia virus in the preparation of an anti-tumor drug. The present disclosure has found for the first time that combined use of the oncolytic vaccinia virus and DR-18 has a synergistic anti-tumor effect. The present disclosure also relates to an anti-tumor pharmaceutical composition comprising an oncolytic vaccinia virus and DR-18, and use of an oncolytic vaccinia virus and DR-18 in the preparation of an anti-tumor drug.

The present application claims the benefits of CN202211566283.7 filed on Dec. 7, 2022, the contents of which are incorporated herein by reference in their entireties.

### FIELD

The present disclosure belongs to the field of biological medicines, and relates to use of DR-18 and an oncolytic vaccinia virus in the preparation of an anti-tumor drug.

### BACKGROUND

Malignant tumor is the first primary fatal disease threatening human health. According to data of “Global Cancer Statistics”, there were 19.29 million new cancer cases and 9.95 million deaths due to cancer worldwide (SUNG H, FERLAY J, SIEGEL R L, et al. Global cancer statistics 2020: GLOBOCAN estimates of incidence and mortality worldwide for 36 cancers in 185 countries [J]. CA Cancer J Clin, 2021, 0: 1-41.). It is expected that by 2040, the number of new cancer cases worldwide will reach 28.4 million, an increase of 47% over 2020, and least developed or developing countries will have the largest increase in cases, 95% and 64% respectively (Cao Maomao, and Chen Wanqing. Interpretation of GLOBOCAN 2020 global cancer statistics [J]. Chinese Journal of Medical Frontiers (Electronic Edition), 2021, 13(3): 63-69.). Therefore, the general public has a very wide demand for efficient and economical anti-tumor drugs (Wanqing Chen, Rongshou Zheng, et al. Cancer Statistics in China, 2015 [J]. CA Cancer J Clin, 2016, 6(6): 115-132.).

Conventional treatment means for malignant tumors include surgery, radiotherapy, and chemotherapy, which have made significant progress in the past few decades. However, these treatment means still cannot substantially improve the long-term survival rate of patients, especially, patients with refractory tumors such as liver cancer, glioma, pancreatic cancer, and osteosarcoma. Cytotoxic drugs and molecular targeted drugs are currently used as clinical first-line anti-tumor drugs, but have the limitations of great toxic and side effects and/or susceptibility to drug resistance. Therefore, the treatment of malignant tumors urgently requires novel efficient and low-toxic treatment means and drugs.

Currently, immunotherapy is a highly anticipated cancer treatment method. Different from direct action of the conventional surgery, radiotherapy, chemotherapy, and targeted drugs, immunotherapy kills a tumor by activating the patient's own immune system, so it has little impact on the normal tissue. Immunotherapy is still effective in some advanced tumors, and can even prevent tumor recurrence to achieve complete cure. Currently, the following clinical drugs have been approved by the U.S. Food and Drug Administration (FDA) for cancer immunotherapy: 1) immune checkpoint inhibitors, such as PD-1 antibodies (Nivolumab, Pembrolizumab, and the like), PD-L1 antibodies (Atezolizumab), and CTLA4 antibodies (Ipilimumab); 2) a plurality of monoclonal antibodies targeting surface tumor-associated antigens, such as anti-CD20 monoclonal antibodies (Rituximab); 3) two immune stimulating cytokines (interferon IFN-α) and interleukin 2 (IL-2); 4) immunogenic cell death inducers, such as Cyclophosphamide and Oxaliplatin; 5) chimeric antigen receptor T-cell therapy (CART); 6) bacille Calmette-Guerin (BCG); 7) dendritic cell-based cancer vaccines; 8) oncolytic viruses (OVs), and the like.

Oncolytic virus therapy is a novel anti-tumor immunotherapy combining targeted therapy, immunotherapy, and gene therapy, can selectively infect and directly kill tumor cells, and then activate anti-tumor immune response by the mechanism, such as exposure of a tumor/viral antigen and release of cytokines, to play an anti-tumor role directly or indirectly. In addition, an oncolytic virus itself may serve as a vector to carry a gene such as a suicide gene, an immune regulatory gene, an apoptosis-promoting gene or an anti-angiogenesis gene, so as to further regulate a tumor microenvironment and promote the anti-tumor efficacy. Compared with the current clinical conventional treatment methods, oncolytic virus therapy has the advantages of strong killing effect, high safety, low cost, and the like.

Because oncolytic virus therapy has unique multiple anti-tumor action pathways, many teams of scholars are dedicated to research and development of such therapy. However, only four oncolytic virus products have been approved for marketing and applied in the clinical treatment of tumors so far, which are RIGVIR approved for marketing in Latvia in 2003, Oncorine (H101) approved for marketing in China in 2005, IMLYGIC (T-Vec) approved for marketing in USA in 2015, and Delytact approved for marketing in Japan in 2021. In addition, several oncolytic viruses are also in clinical trials, among which adenovirus (ADV), herpes simplex virus (HSV), and vaccinia virus (VV) are the most widely used clinically.

Vaccinia virus is a relatively well-studied virus. It was once used as a smallpox vaccine and had been widely vaccinated worldwide. It has a history of nearly 200 years and has shown good effects and safety. Due to its advantages of low disease-causing risk, clear pathogenicity and pathogenic gene, mature attenuation strategy, stable genome, non-integration, non-latency, strong oncolytic effect, activation of immune response, large load and stable expression of exogenous genes, intravenous administration, and the like, vaccinia virus has been widely recognized as one of the ideal engineered oncolytic virus skeletons. Based on the foregoing development advantages, a number of clinical trials of oncolytic vaccinia viruses have been carried out in China and other countries, which demonstrate good safety and efficacy. For example, JX-594 has been approved for a phase III international multi-center clinical trial in China.

Although in recent decades, oncolytic vaccinia viruses that modified by artificial genetic modification have made great progress in the field of tumor treatment, so far no therapeutic oncolytic vaccinia virus product has been successfully approved for marketing. Vaccinia virus strains in clinical trials include WR, Lister, Copenhagen, Wyeth, LC16m0, MVA, and the like. Artificial genetic modification strategies are as follows: T601/TG6002 is obtained by knocking out the TK and RR genes from Copenhagen and loading the FCU1 gene; ASP9801 is obtained by knocking out the VGF, 11L, and B5R genes from LC16m0 and loading the IL-7 and IL-12 genes; and JX-594 is obtained by knocking out the TK gene from Wyeth and loading the GM-CSF gene. There is still a lot of room for progress in the research on the mechanism of tumor-killing effect of an oncolytic vaccinia virus and application thereof. For example, issues, such as elimination or escape of antiviral antibodies, development of immunotherapy targets, combined administration of an oncolytic virus and another anti-cancer drug, administration routes, and indications, still require more research.

IL-18 is an immune-activating cytokine that can simulate T cells, NK cells, and bone marrow cells and has the ability to activate anti-tumor immune cells, and thus may serve as a candidate molecule for treating cancer. However, IL-18BP (IL-18 binding protein) in a tumor microenvironment may serve as a secreted immune checkpoint molecule, and competitively binds to IL-18 and blocks binding of IL-18 to a receptor, which limits the effect of IL-18 immunotherapy. Therefore, immune cells cannot activate anti-tumor immune response.

### SUMMARY

In an aspect, the present disclosure provides DR-18 for use in treating tumor by administering DR-18 in combination with oncolytic vaccinia virus.

In an aspect, the present disclosure provides oncolytic vaccinia virus for use in treating tumor by administering said oncolytic vaccinia virus in combination with DR-18.

In an aspect, the present disclosure provides use of DR-18 in the preparation of a medicament for treating tumor to be used in combination with an oncolytic vaccinia virus. In an aspect, the present disclosure provides use of an oncolytic vaccinia virus in the preparation of a medicament for treating tumor to be used in combination with DR-18.

In an aspect, the present disclosure provides of DR-18 in the preparation of an oncolytic vaccinia virus anti-tumor synergist or drug resistance reversal agent. In another aspect, the present disclosure provides use of an oncolytic vaccinia virus in the preparation of a DR-18 anti-tumor synergist or drug resistance reversal agent.

A drug resistance reversal agent refers to that when some oncolytic viruses are employed as anti-tumor drugs for treating tumors, there exist some tumors that are not very sensitive to the oncolytic viruses or that are resistant to the oncolytic viruses, and in this case, combined use of IL-18 (serves as a drug resistance reversal agent) and the oncolytic viruses can reverse the resistance of the tumors to the oncolytic viruses. Or, conversely, when some anti-tumor substances are used for treating tumors, there exist some tumors that are not very sensitive to the drugs or that are resistant to these substances, and in this case, combined use of an oncolytic virus (serves as a drug resistance reversal agent) and these substances can reverse the resistance of the tumors to the substances.

DR-18 is mutated IL-18, which binds to and activates an IL-18 receptor and a downstream pathway thereof, and does not bind to IL-18BP. For example, the U.S. patent application No. 2019/0070262 and Zhou et al, (Nature (2020) 583: 609-614) disclose some human DR-18 and some murine DR-18.

In some embodiments, DR-18 is human DR-18. In some embodiments, human DR-18 comprises at least one mutation relative to wild-type human IL-18.

In some embodiments, human DR-18 comprises one or more of the following mutations relative to wild-type human IL-18 selected from the group consisting of M51K, K53S, Q56L, P57A, M60L, S105D, D110S, and N111R. In some embodiments, human DR-18 comprises the following mutations relative to wild-type human IL-18: M51K, K53S, Q56L, P57A, M60L, S105D, D110S, and N111R. In some embodiments, an amino acid sequence of human DR-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 1. In some embodiments, an amino acid sequence of wild-type human IL-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 3.

In some embodiments, DR-18 is murine DR-18. In some embodiments, murine DR-18 comprises at least one mutation relative to wild-type murine IL-18.

In some embodiments, murine DR-18 comprises one or more of the following mutations relative to wild-type murine IL-18 selected from the group consisting of N1H, M50A, K52G, E55R, V56A, and L59K. In some embodiments, murine DR-18 comprises the following mutations relative to wild-type murine IL-18: N1H, M50A, K52G, E55R, V56A, and L59K.

In some embodiments, an amino acid sequence of murine DR-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 2. In some embodiments, an amino acid sequence of wild-type murine IL-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 4.

In some embodiments, the oncolytic vaccinia virus is a type with a defective TK gene. In some embodiments, the TK gene of the oncolytic vaccinia virus is inactivated, under-expressed or deleted. In some embodiments, the TK gene of the oncolytic vaccinia virus is deleted. In some embodiments, the oncolytic vaccinia virus is one or more selected from the group consisting of a WR strain, a Wyeth strain, a Lister strain, a Copenhagen strain, and a Tiantan strain. In some embodiments, the oncolytic vaccinia virus is selected from a Wyeth strain.

As used in the present disclosure, “human DR-18” and DR18 are interchangeable, and “murine DR-18” and “mDR18” are interchangeable.

As used in the present disclosure, “mutation”, “mutant”, or “variant” refers to an alteration in a nucleic acid or polypeptide sequence relative to a reference sequence (the reference sequence may be a naturally occurring normal or “wild-type” sequence), and includes translocation, deletion, insertion, and substitution/point mutation. “Mutant” or “variant” used herein refers to a nucleic acid or protein containing a mutation.

As used in the present disclosure, the term “wild type” refers to a gene or gene product isolated from a natural source. A wild-type gene is the most commonly observed gene in the population, and thus arbitrarily designated as the “normal” or “wild-type” form of the gene. On the contrary, the term “modified”, “variant” or “mutant” refers to a gene or gene product with a sequence and/or functional characteristic modification (that is, an altered characteristic) relative to a wild-type gene or gene product.

As used in the present disclosure, the term “functional defect” used to refer to a gene of an oncolytic virus refers to that the oncolytic virus cannot perform the function as intended by the gene, namely, a loss of function, which may be realized by (for example) inserting an exogenous fragment into the gene or knocking out the gene.

In some embodiments, a functional defect of a gene may be realized by inserting an exogenous nucleotide sequence into the gene and/or knocking out the gene.

In an aspect, the present disclosure provides a combination of DR-18 and oncolytic vaccinia virus for treating tumors. In some embodiments, “combination” is interpreted broadly, for example, as a pharmaceutical composition, a medicine kit, and a combination of DR-18 genes with oncolytic vaccinia virus genes at the molecular level.

In an aspect, the present disclosure provides a pharmaceutical combination for treating a tumor comprising: DR-18, and an oncolytic vaccinia virus.

The term “pharmaceutical combination” as used herein means a product that results from the mixing or combining of more than one active ingredient and includes both fixed and non-fixed combinations of the active ingredients. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “fixed combination” as that term is used herein means that the active ingredients, e.g., DR-18 and an oncolytic vaccinia virus, are both administered to a patient simultaneously in the form of a single entity or dosage.

The term “non-fixed combination” as that term is used herein means that the active ingredients, e.g. DR-18 and an oncolytic vaccinia virus, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

The term “combined use” or “use in combination” as used herein means that two or more active substances may be administered to a subject as a mixture, simultaneously as a single formulation, or sequentially in any order as a single formulation.

The term “pharmaceutical combination” as used herein comprises that the target gene (in this disclosure, for example, DR18) is recombined into the oncolytic vaccinia virus genome, resulting in a recombinant oncolytic vaccinia virus that can exert pharmaceutical activity of both oncolytic vaccinia virus and DR18.

The term “in combination with” as used herein when used in reference to the administration of multiple agents to a subject refers to the administration of a first agent at least one additional (i.e. second, third, fourth, fifth, etc.) agent to a subject. For purposes of the present invention, one agent (e.g. DR-18) is considered to be administered in combination with a second agent (e.g. an oncolytic vaccinia virus) if the biological effect resulting from the administration of the first agent persists in the subject at the time of administration of the second agent such that the therapeutic effects of the first agent and second agent overlap. The administration of the first agent (e.g. DR-18) provides a therapeutic effect over an extended time and the administration of the second agent (e.g. an oncolytic vaccinia virus) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In some embodiments, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also be understood to apply to the situation where a first agent and a second agent are co-formulated in single pharmaceutically acceptable formulation and the co-formulation is administered to a subject. In certain embodiments, the DR-18 and the oncolytic vaccinia virus(s) are administered or applied sequentially, e.g., where one agent is administered prior to one or more other agents. In other embodiments, the DR-18 and the oncolytic vaccinia virus(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

The term “about” or “approximately” used herein shall have the meaning of within 10%, more preferably within 5%, of a given value or range.

In some embodiments, the pharmaceutical combination is a pharmaceutical composition or a medicine kit. In some embodiments, the pharmaceutical composition comprises the mixture of DR-18, and an oncolytic vaccinia virus. In some embodiments, the medicine kit comprises independently packaged DR-18 and an independently packaged oncolytic vaccinia virus. In some embodiments, the pharmaceutical combination is a recombinant oncolytic vaccinia virus with DR-18 nucleotide sequence inserted in the genome.

In an aspect, the present disclosure provides a pharmaceutical composition for treating a tumor, which comprises:
DR-18, and an oncolytic vaccinia virus.

In an aspect, the present disclosure provides a medicine kit, which comprises:
DR-18, and an oncolytic vaccinia virus.

In some embodiments, the medicine kit comprises independently packaged DR-18 and an independently packaged oncolytic vaccinia virus.

A difference between the medicine kit and the pharmaceutical composition is that DR-18 is in a dosage form different from that of the oncolytic vaccinia virus, and is independently packaged (for example, a pill, capsule, tablet or ampoule bottle contains DR-18, and another pill, capsule, tablet or ampoule bottle contains the oncolytic vaccinia virus). In some embodiments, the oncolytic vaccinia virus, DR-18, and a combination of the oncolytic vaccinia virus and DR-18 may also contain one or more adjuvants. The adjuvant refers to an ingredient in a pharmaceutical composition that can assist in the efficacy of the drug. The medicine kit may also contain independently packaged DR-18, and an independently packaged oncolytic vaccinia virus. DR-18 and the oncolytic vaccinia virus in the medicine kit may be administered simultaneously or administered successively in any order. For example, DR-18 is administered before the oncolytic vaccinia virus, or DR-18 is administered after the oncolytic vaccinia virus, or DR-18 and the oncolytic vaccinia virus are administered simultaneously. In various embodiments, a patient may be a mammal.

In some embodiments, the composition/medicine kit further comprises a pharmaceutically acceptable vector.

In some embodiments, a ratio of DR-18 to the oncolytic vaccinia virus is 0.01-200 mg: 10³-10⁹ PFU, preferably 0.1-200 mg: 10⁴-10⁹ PFU, and more preferably 0.1-100 mg: 10⁵-10⁹ PFU.

In some embodiments, doses are as follows: a dose range of DR-18 is 0.01-10 mg/kg, and titers of the oncolytic vaccinia virus are MOI 10³-10⁹ PFU/kg, preferably, the dose range of DR-18 is 0.1-5 mg/kg, and the titers of the oncolytic vaccinia virus are MOI 10⁴-10⁹ PFU/kg, and more preferably, the dose range of DR-18 is 0.05-0.5 mg/kg, and the titers of the oncolytic vaccinia virus are MOI 10⁵-10⁹ PFU/kg.

In some embodiments, DR-18 is administered by intraperitoneal injection. In some embodiments, the oncolytic vaccinia virus is administered by intratumor injection or intravenous injection. In some embodiments, the form of the pharmaceutical combination provided by the present disclosure comprises injection, tablet, capsule, or patch, etc. In some embodiments, the form of the pharmaceutical combination of the present disclosure is an intratumoral injection, an intraperitoneal injection or an intravenous injection.

In some embodiments, the tumor is a solid tumor or blood tumor.

In some embodiments, the solid tumor is one or more selected from the group consisting of bowel cancer, pancreatic cancer, liver cancer, bladder cancer, breast cancer, cervical cancer, prostate cancer, glioma, melanoma, nasopharyngeal cancer, lung cancer, osteosarcoma, and gastric cancer.

In an aspect, the present disclosure provides use of DR-18 in the preparation of a combination therapy for treating tumor, wherein the combination therapy comprises an oncolytic vaccinia virus in combination with DR-18.

In an aspect, the present disclosure provides use of an oncolytic vaccinia virus in the preparation of a combination therapy for treating tumor, wherein the combination therapy comprises DR-18 in combination with an oncolytic vaccinia virus.

In an aspect, the present disclosure provides use of the pharmaceutical combination, the pharmaceutical composition, the medicine kit, or the combination therapy in preparation of an anti-tumor drug.

In an aspect, the present disclosure provides a recombinant oncolytic vaccinia virus. A genome of the recombinant oncolytic vaccinia virus comprises: a mutated oncolytic vaccinia virus sequence, and a DR-18 sequence.

In some embodiments, the mutation is a functional defect of the TK gene. In some embodiments, the mutation is inactivation, under-expression or deletion of the TK gene. In some embodiments, the mutation is deletion of the TK gene. In some embodiments, the DR-18 sequence is a human DR-18 sequence and/or a murine DR-18 sequence.

In some embodiments, a nucleotide sequence of human DR-18 comprises a nucleotide sequence having at least 85%, at least 90%, at least 91%, at least 92%, at least 93%, at least 94%, at least 95%, at least 96%, at least 97%, at least 98%, at least 99%, or 100% identity to the nucleotide sequence as shown in SEQ ID NO: 5.

In some embodiments, a nucleotide sequence of murine DR-18 comprises a nucleotide sequence having at least 85%, at least 90%, at least 91%, at least 92%, at least 93%, at least 94%, at least 95%, at least 96%, at least 97%, at least 98%, at least 99%, or 100% identity to the nucleotide sequence as shown in SEQ ID NO: 6.

In an aspect, the present disclosure provides a pharmaceutical composition, which comprises the recombinant oncolytic vaccinia virus, and a pharmaceutically acceptable vector.

In some embodiments, the pharmaceutical composition comprises 10⁴-10⁶ PFU of recombinant oncolytic vaccinia virus. In some embodiments, the pharmaceutical composition comprises 10⁴-10⁵ PFU of recombinant oncolytic vaccinia virus. In some embodiments, the pharmaceutical composition comprises 10⁵-10⁶ PFU of recombinant oncolytic vaccinia virus.

The present disclosure further provides use of the pharmaceutical combination, the recombinant oncolytic vaccinia virus, or the pharmaceutical composition in the preparation of an anti-tumor drug.

In an aspect, the present disclosure provides a method for the prevention and/or treatment of a tumor, which comprises administering to a subject in need thereof the pharmaceutical combination, the recombinant oncolytic vaccinia virus, or the pharmaceutical composition. The DR-18 may be administered concurrently, before, or subsequent to, administration of an oncolytic vaccinia virus contemplated herein. Additionally, the DR-18 and/or the oncolytic vaccinia virus may be administered once a week, or several times (e.g., 2, 3, 4, 5, 6, 7, 8, 9 or 10) a week. The DR-18 and/or the oncolytic vaccinia virus may be administered for one or several weeks (1, 2, 3, 4, 5, 6, 7, 8, 9, or 10), for a month, or even for several months (2, 3, 4, 5, 6, 7, 8, 9, 10, 11 or 12 or more).

In some embodiments, combinations of some oncolytic viruses and DR-18 have achieved various unforeseen effects. The inventors have found that some combinations have obvious mutual antagonism or inhibition. However, in contrast to these phenomena, combined use of oncolytic viruses and DR-18 show obvious synergetic effects in tests on various tumors, which is a surprising and promising finding.

### BRIEF DESCRIPTION OF THE DRAWINGS

FIG. 1 shows a gene structure of a plasmid 1 in which the TK gene is knocked out and that carries the LacZ reporter gene of Example 1;

FIG. 2 shows a gene structure of a plasmid 2 in which the TK gene is knocked out and that carries the GFP gene of Example 1;

FIG. 3 shows a gene structure of a plasmid 3 in which the TK gene is knocked out and that expresses the mouse DR18 gene (SEQ ID NO: 6) of Example 1;

FIG. 4 is a diagram of a gene structure of a recombinant oncolytic vaccinia virus in which the TK gene is knocked out of Example 2 and Example 3;

FIG. 5 is a diagram of a gene structure of a recombinant oncolytic vaccinia virus in which the TK gene is knocked out of Example 4;

FIG. 6 is a diagram of a gene structure of a recombinant oncolytic vaccinia viruses VV-mDR18 in which the TK gene is knocked out and that expresses the mouse DR18 gene of Example 4 and Example 5;

FIG. 7 shows influences of OVs-SN on the activity of DR18 of Example 2, and in the figure, * denotes P<0.05, ** denotes P<0.01, *** denotes P<0.001, and n.s. denotes a statistically non-significant difference;

FIG. 8 shows body weight change curves of animals treated with combinations of OVs and mDR18 of Example 3, and in the figure, n.s. denotes a statistically non-significant difference;

FIG. 9 shows a tumor volume growth curve of a VV+mDR18 group of Example 3, and in the figure, * denotes P<0.05, ** denotes P<0.01, *** denotes P<0.001, and n.s. denotes a statistically non-significant difference;

FIG. 10 shows a tumor volume growth curve of an ADV+mDR18 group of Example 3, and in the figure, * denotes P<0.05, ** denotes P<0.01, *** denotes P<0.001, and n.s. denotes a statistically non-significant difference;

FIG. 11 shows a tumor volume growth curve of a VSV+mDR18 group of Example 3, and in the figure, * denotes P<0.05, ** means P<0.01, *** means P<0.001, and n.s. denotes a statistically non-significant difference;

FIG. 12 shows body weight change curves of animals treated with different doses of VV-mDR18 of Example 4, and in the figure, n.s. denotes a statistically non-significant difference;

FIG. 13 shows tumor volume growth curves of animals treated with different doses of VV-mDR18 of Example 4, and in the figure, *** denotes P<0.001;

FIG. 14 shows relative tumor growth rate curves of animals treated with different doses of VV-mDR18 of Example 4;

FIG. 15 shows body weight change curves of animal models of different tumors treated with VV-mDR18 of Example 5, and in the figure, A represents an MC38 tumor-bearing mouse model of bowel cancer, B represents an LLC tumor-bearing mouse model of lung cancer, and C represents an H22 tumor-bearing mouse model of liver cancer;

FIG. 16 shows tumor volume growth curves of models of different tumors treated with VV-mDR18 of Example 5 (** denotes P<0.01), and in the figure, A represents an MC38 tumor-bearing mouse model of bowel cancer, B represents an LLC tumor-bearing mouse model of lung cancer, and C represents an H22 tumor-bearing mouse model of liver cancer; and

FIG. 17 shows relative tumor growth rate curves of models of different tumors treated with W-mDR18 of Example 5, and in the figure, A represents an MC38 tumor-bearing mouse model of bowel cancer, B represents an LLC tumor-bearing mouse model of lung cancer, and C represents an H22 tumor-bearing mouse model of liver cancer.

FIG. 18 shows influences of inactivated supernatants of different tumor cells infected with various oncolytic viruses on the activity of DR18 (* denotes P<0.05, ** denotes P<0.01, and *** denotes P<0.001).

### DETAILED DESCRIPTION

The present disclosure will be further described below with reference to embodiments. However, the embodiments of the present disclosure are not limited to the following embodiments. All equivalent changes or modifications made in accordance with the principles or concepts of the present disclosure shall be deemed to fall within the scope of protection of the present disclosure. Unless otherwise specified, materials and experimental methods adopted in the present disclosure are conventional materials and methods.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Use of words such as “approximate”, “about”, “substantially”, etc. modifies the value/description as would be understood by a person of skill in the art based upon the context and the parameter being described, and where further guidance is needed in order to be understood, a value of 5% can be applied. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” Described percent sequence identity refers to the percentage of nucleic acid or amino acid residues within a given DNA, RNA or protein, respectively, that are identical to the reference sequence.

### Example 1 Construction of Recombinant Oncolytic Vaccinia Viruses in which the TK Gene is Knocked Out and Recombinant Oncolytic Vaccinia Viruses in which the TK Gene is Knocked Out and that Express the Mouse DR18 Gene

### 1. Experimental Materials

1.1 Experimental viruses: a wild vaccinia virus Wyeth strain was purchased from the American Type Culture Collection (ATCC).
1.2 Experimental plasmids: a TK gene, an LacZ gene or a GFP gene, and a murine DR18 gene were synthesized, and these genes were recombined into a pUC57-Mini plasmid by strain design. The plasmid was purchased from GenScript.

A plasmid 1 in which the TK gene was knocked out and that carried the LacZ reporter gene, a plasmid 2 in which the TK gene was knocked out and that carried the GFP gene, and a plasmid 3 in which the TK gene was knocked out and that expressed the mouse DR18 gene (SEQ ID NO: 6) were constructed.

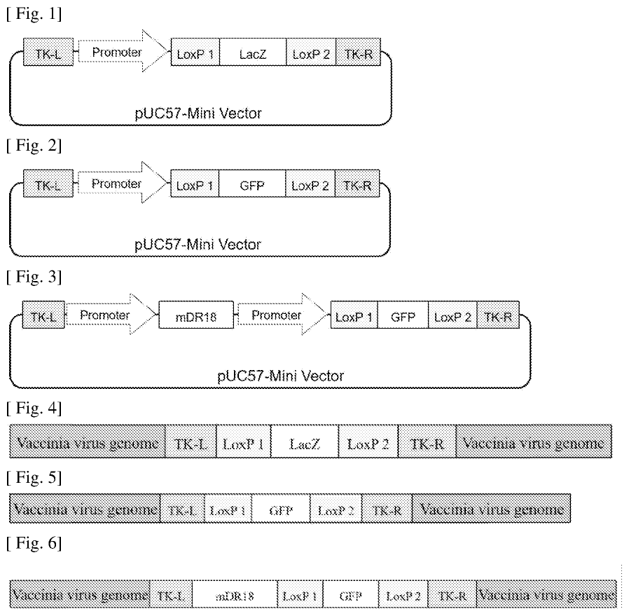

A gene structure of the plasmid 1 in which the TK gene is knocked out and that carries the LacZ reporter gene is shown in FIG. 1.

A gene structure of the plasmid 2 in which the TK gene is knocked out and that carries the GFP gene is shown in FIG. 2.

A gene structure of the plasmid 3 in which the TK gene is knocked out and that expresses the mouse DR18 gene (SEQ ID NO: 6) is shown in FIG. 3.
1.3 Experimental cells: human thymidine kinase deficient osteosarcoma cells (143B (TK-)) were purchased from ATCC.

### 2. Experimental Method

2.1 Homologous recombination

One day before the experiment, the 143B (TK-) cells were inoculated into a cell culture flask and cultured overnight. A viral solution of the wild vaccinia virus Wyeth strain was taken to infect the cells. According to the instruction for use of a Lipofectamine™ 3000 transfection reagent, a plasmid transfection system was prepared based on a usage amount of recombinant plasmid, and added to the cells. After the cytopathic effect reached more than 80%, a recombinant viral solution was collected.
2.2 Plaque purification

One day before the experiment, the 143B (TK-) cells were inoculated into a 12-well plate and cultured overnight. The recombinant viral solution was subjected to 10-fold gradient serial dilution, and added to the cells for incubation for 60 min. A virus-infected supernatant was removed, a 1% low-melting-point agarose-5% FBS MEM solid medium was added for solidification at room temperature, and the cells were cultured for 24-48 h. A solid medium containing X-gal was added to the 12-well plate for solidification at room temperature, and a color was developed with X-gal for 24-48 h. A blue plaque was punctured by using a pipette tip, frozen and thawed repeatedly, and used for the next plaque purification. The number of times of plaque purification was not less than 5. Virus clones subjected to plaque purification were amplified to obtain a recombinant oncolytic vaccinia virus seed strain.

### Example 2 Evaluation of Influences of Supernatants of Different Tumor Cells Infected with Various Oncolytic Viruses on the Activity of DR 18 by Using an IL-18 Reporter Cell Model

IL-18 reporter cell model: this cell expresses an IL-18 receptor, an IL-18 downstream pathway molecule, and luciferase, IL18 binds to a receptor and mediates the expression of luciferase, and the cell count represents the pathway activating effect. The pathway is a key pathway for DR18 to activate anti-tumor immunity in vivo, and can reflect the anti-cancer activity of DR18 indirectly.

### 1. Experimental Materials

1.1 Oncolytic viruses: VV: it is a recombinant oncolytic vaccinia virus in which the TK gene is knocked out, and a genome structure of the oncolytic vaccinia virus is shown in FIG. 4.

ADV: it is an oncolytic adenovirus in which the E1a-CR2-24 bp gene and genes in the E3 region are knocked out. VSV: it is an attenuated oncolytic vesicular stomatitis virus in which the G gene is knocked out and the M gene has V48R&M51R mutations. REO: it is a serotype 3 oncolytic reovirus. The viruses were products on the market, or customized products constructed by conventional methods, or constructed by Guangzhou ViroTech Co., Ltd. by conventional methods.
1.2 Experimental cells: IL-18 reporter cells (H_IL18 Reporter 293 Cell Line), prostate cancer cells (DU145), bladder cancer cells (SCaBER and KU-19-19), pancreatic cancer cells (MIA Paca-2), kidney cancer cells (786-0), cervical cancer cells (HCC94 and C-33A), bowel cancer cells (DLD-1), liver cancer cells (Hep3B), lung cancer cells (SHP-77 and A549), breast cancer cells (HCC38), and osteosarcoma cells (MNNG/HOS) were purchased from the market.
1.3 Experimental reagents: human DR18 recombinant protein (SEQ ID NO: 1), a DMEM medium, an MEM medium, a RPMI 1640 medium, a McCoy's 5A medium, an IMDM medium, an EMEM medium, Fetal Bovine Serum, Puromycin, Blasticidin, G418, and ONE-Glo™ Luciferase Assay System.
1.4 Experimental instruments: an inverted microscope, a biosafety cabinet, a carbon dioxide incubator, and a microplate reader.

### 2. Experimental Method

2.1 Construction of tumor cell models

According to the instruction for use of the tumor cells, appropriate culture conditions and a passage proportion were selected for cell proliferation and passage. After the cells entered the logarithmic growth phase and the number of cells was sufficient, the cells were inoculated into a T25 cell culture flask according to a density of 3×10⁴ cells/cm².
2.2 Infection of tumor cells with oncolytic viruses

16-24 h after the tumor cells were inoculated, complete attachment of the cells was observed under the microscope. According to the number of cells plated, the tumor cells were infected with a viral load of MOI=1 PFU/cell.
2.3 Collection and inactivation of infected supernatants

After the tumor cells were infected with the oncolytic virus for 48 h, a cytopathic effect was observed under the microscope, and photographed and recorded. According to the characteristics of the virus, a virus-infected cell supernatant (SN) was inactivated by ultraviolet radiation or filtered with a 0.1 μm filter membrane, and the inactivated supernatant was collected into 4.5 mL cryogenic vials and stored in a refrigerator at −80° C. for later use. An uninfected supernatant was collected by the same method and taken as a control.
2.4 Influences of infected supernatants on the biologic activity of DR18
1) The IL-18 reporter cells were thawed and cultured for passage. After the cells entered the logarithmic growth phase and the number of cells was sufficient, the cells were inoculated into middle wells in a 96-well plate according to a density of 1.5×10⁴ cells/well, and 90 μL of medium was added to each middle well, and 200 μL of PBS was added to surrounding wells for edge sealing.
2) The cells were cultured in the cell incubator with 5% CO₂ at 37° C. for 16-24 h, the 96-well plate was taken out, and complete attachment and good morphology of the cells were observed under the microscope. The DR18 protein was subjected to 3-fold proportional gradient dilution with the inactivated supernatants and the control supernatants obtained in step 2.3, and after being mixed uniformly, the mixture was added to the cells according to an addition amount of 10 μL/well.
3) After the DR18 protein was added, the cells were cultured in the cell incubator with 5% CO₂ at 37° C. for 16 h.
4) 100 μL of ONE-Glo™ substrate was added and reacted in the dark for 3 min to allow complete cell lysis, the cell lysis solution was transferred into a 96-well luminescent plate, and luciferase was detected by using the microplate reader.
2.5 Data processing

Based on detected relative light units (RLU) in each well, a relative light rate of the cells in each well was calculated by using the following formula: relative light rate (% of Max)=(RLUₛₐₘₚₗₑ−RLU_{blank})/(RLUₘₐₓ−RLU_{blank})×100%. A fitted curve was drawn by using GraphPad Prism 8 software. A half maximal effective concentration (EC₅₀) value of the biological activity of DR18 was calculated by using the [Agonist] vs. response—Variable slope (four parameters) analytical equation.
2.6 Biological statistics

The detection data was subjected to statistical analysis by T tests. * denotes P<0.05, ** denotes P<0.01, and *** denotes P<0.001, which all indicate that a difference between data is statistically significant.

### 3. Experimental Results

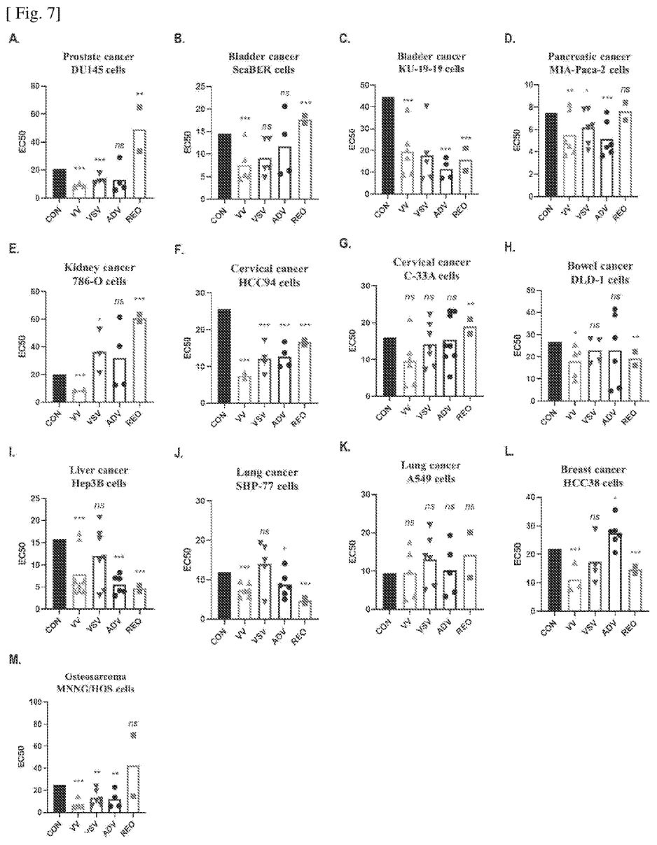

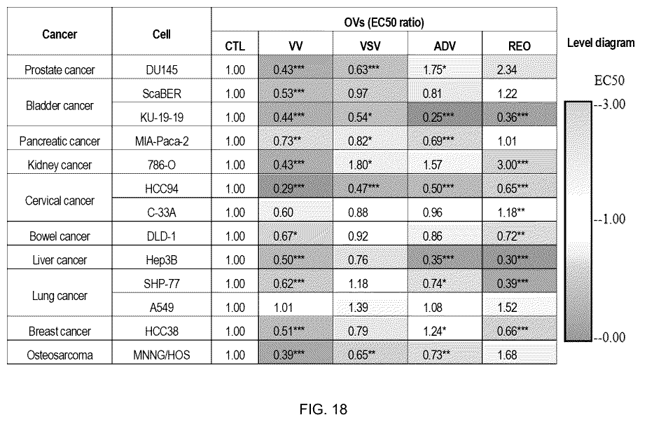

3.1 Influences of the inactivated supernatants of different tumor cells infected with various oncolytic viruses on the activity of DR18 are shown in FIG. 7 and FIG. 18.

The DR18 recombinant protein was subjected to gradient dilution with the supernatants of different tumor cells infected with different oncolytic viruses, the IL-18 reporter cells were treated for 16 h, the light units of luciferase were detected by the machine, and the fitted curve was drawn. The calculated EC₅₀ values of the treatment groups are shown in FIG. 7, and the statistical analysis results are shown in FIG. 18.

The results show that compared with the uninfected control group (CTL), oncolytic vaccinia virus (VV) infection of the prostate cancer cells (DU145), the bladder cancer cells (SCaBER and KU-19-19), the pancreatic cancer cells (MIA-Paca-2), the kidney cancer cells (786-0), the cervical cancer cells (HCC94), the bowel cancer cells (DLD-1), the liver cancer cells (Hep3B), the lung cancer cells (SHP-77), the breast cancer cells (HCC38), and osteosarcoma cells (MNNG/HOS) can significantly promote the biological activity of DR18. Oncolytic vaccinia virus (VV) infection of the cervical cancer cells (C-33A) tends to promote the biological activity of DR18, but does not have a statistically significant difference.

Oncolytic vesicular stomatitis virus (VSV) infection of the prostate cancer cells (DU145), the bladder cancer cells (KU-19-19), the pancreatic cancer cells (MIA Paca-2), the cervical cancer cells (HCC94), and the osteosarcoma cells (MNNG/HOS) can significantly promote the biological activity of DR18. On the contrary, oncolytic vesicular stomatitis virus (VSV) infection of the kidney cancer cells (786-0) inhibits the biological activity of DR18.

Oncolytic adenovirus (ADV) infection of the bladder cancer cells (KU-19-19), the pancreatic cancer cells (MIA Paca-2), the cervical cancer cells (HCC94), the liver cancer cells (Hep3B), the lung cancer cells (SHP-77), and the osteosarcoma cells (MNNG/HOS) can significantly promote the biological activity of DR18. On the contrary, oncolytic adenovirus (ADV) infection of the prostate cancer cells (DU145) and the breast cancer cells (HCC38) inhibits the biological activity of DR18.

Oncolytic reovirus (REO) infection of the bladder cancer cells (KU-19-19), the cervical cancer cells (HCC94), the bowel cancer cells (DLD-1), the liver cancer cells (Hep3B), the lung cancer cells (SHP-77), and the breast cancer cells (HCC38) can significantly promote the biological activity of DR18. On the contrary, oncolytic reovirus (REO) infection of the kidney cancer cells (786-0) and the cervical cancer cells (C-33A) inhibits the biological activity of DR18.

It can be seen from the experimental results that the influences of the supernatants of different tumor cell models infected with various oncolytic viruses are different, and the synergistic effect of VV is higher than that of other oncolytic viruses.

### Example 3 Study on the Safety and Efficacy of Combined Used of mDR18 and Various Oncolytic Viruses in Immunocompetent Mouse Models of Liver Cancer

### 1. Experimental Materials

1.1 Oncolytic viruses: VV: it is a recombinant oncolytic vaccinia virus in which the TK gene is knocked out, and a genome structure of the oncolytic vaccinia virus is shown in FIG. 4.

ADV: it is an oncolytic adenovirus in which the E1a-CR2-24 bp gene and genes in the E3 region are knocked out. VSV: it is an attenuated oncolytic vesicular stomatitis virus in which the G gene is knocked out and the M gene has V48R&M51R mutations. The viruses were purchased from Wuhan BrainVTA, FUBIO, and the like, or constructed by Guangzhou ViroTech Co., Ltd.
1.2 Recombinant protein: modified mouse interleukin-18 (mDR18) recombinant protein (SEQ ID NO: 2).
1.3 Tumor cells: a mouse liver cancer cell line H22 was purchased from China Center for Type Culture Collection.
1.4 Experimental animals: 5-7-week old female Balb/c mice.

### 2. Experimental Method

2.1 Construction of tumor-bearing mouse models

The tumor cells were thawed and cultured for passage. After the cells entered the logarithmic growth phase and the number of cells was sufficient, the cells were collected and counted, and a cells suspension was prepared. The H22 cells were inoculated subcutaneously into the back of the Balb/c mice according to the inoculation quantity of 2×10⁶ cells/mouse.
2.2 Grouping and administration

After tumors were formed, mice whose tumor volume was 100±40 mm³ were selected and randomly divided into 8 groups, namely, a vehicle control group, an mDR18 group, a VV group, a VV+mDR18 group, an ADV group, an ADV+mDR18 group, a VSV group, and a VSV+mDR18 group, 5 mice per group. mDR18 was administered by intraperitoneal injection at a dose of 0.32 mg/kg, twice a week, a total of 4 times. Each oncolytic virus was administered by intratumor injection once at a dose of 2×10⁶ PFU/mouse. The day of tumor inoculation was denoted as DO, and the mice were observed for 14 days after administration.
2.3 Measurement of body weight and tumors of mice

During tumor formation and administration, the body weight and the tumor growth of the mouse were recorded every 3 or 4 days.
2.4 Data processing

The measured data was counted, and a tumor volume growth curve of the mice in each group was drawn.

A formula for calculating tumor volume (TV) is as follows: V=1/2×a×b², where, a and b respectively represent a length and a width of a tumor.

Relative tumor volume (RTV)=Vₜ/V₀, where, V₀ is the tumor volume measured before grouping and administration, and Vₜ is the tumor volume at each measurement.

Relative tumor growth rate T/C (%)=T_{RTV}/C_{RTV}×100%, where, T_{RTV} represents RTV of the single administration or combined administration group of each drug, and C_{RTV} represents RTV of the vehicle control group.
2.5 Statistical analysis

The tumor volume and the body weight of the mouse were subjected to statistical analysis by repeated measures analysis of variance, * denotes P<0.05, ** denotes P<0.01, and *** denotes P<0.001, which all indicate that a difference between data is statistically significant.

### 3. Experimental Results

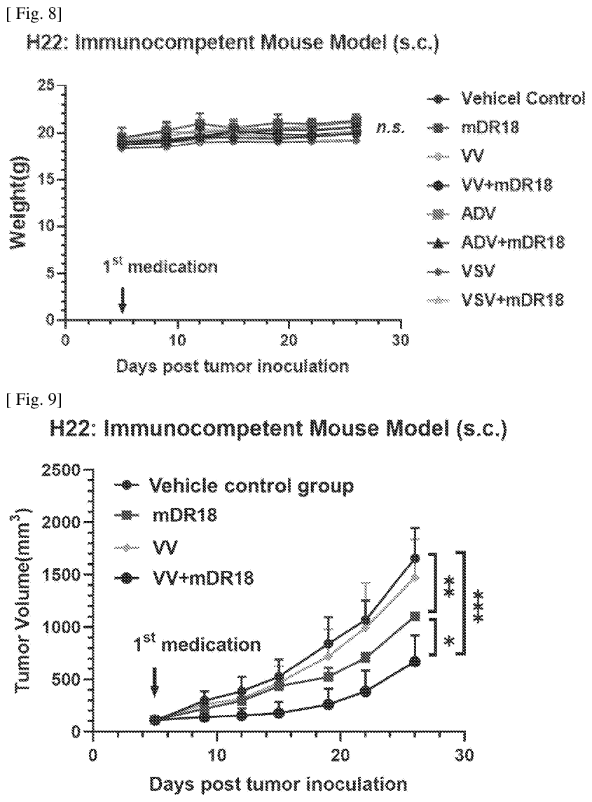

The body weight of the animal in each group during the experiment is shown in FIG. 8. The body weight of the animal in each group maintains a steady upward trend during the experiment. There is no obvious difference between the single administration or combined administration group of each drug and the vehicle control group. It indicates that intraperitoneal injection of mDR18 at a dose of 0.32 mg/kg twice a week or single intratumor injection of each oncolytic virus at a dose of 2×10⁶ PFU/mouse does not cause an obvious drug-associated side effect in the immunocompetent mouse models.

A tumor volume change curve drawn based on the records of the tumor volume growth of the animal is shown in FIG. 9, which is used for evaluating the efficacy. The results show that compared with the vehicle control group, single administration of mDR18 (P<0.01) or combined administration of mDR18 and VV (P<0.001) can significantly inhibit the tumor growth of the tumor-bearing mouse model of liver cancer. Compared with single administration of mDR18, combined administration of mDR18 and VV (P<0.05) can significantly improve the tumor inhibiting effect on the tumor-bearing mouse model of liver cancer.

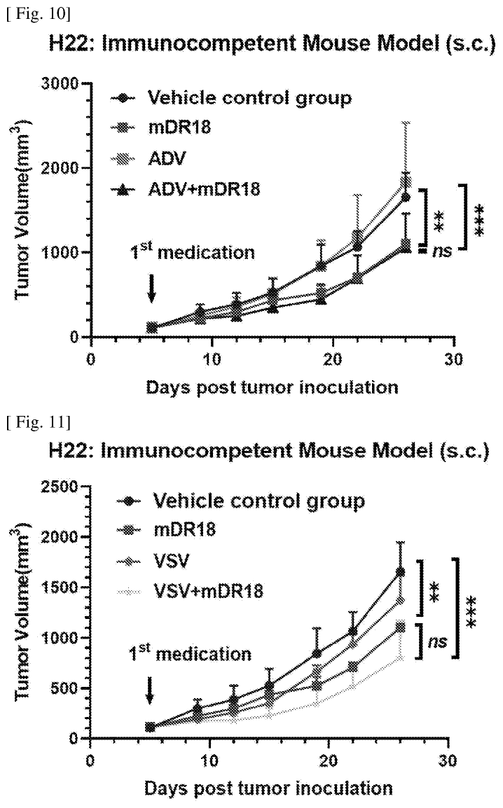

A tumor volume change curve drawn based on the records of the tumor volume growth of the animal is shown in FIG. 10, which is used for evaluating the efficacy. The results show that compared with the vehicle control group, single administration of mDR18 (P<0.01) or combined administration of mDR18 and ADV (P<0.001) can significantly inhibit the tumor growth in the tumor-bearing mouse model of liver cancer. There is no statistically significant difference between the tumor inhibiting effects of single administration of mDR18 and combined administration of mDR18 and ADV, which indicates that combined administration of mDR18 and ADV has no synergistic effect.

A tumor volume change curve drawn based on the records of the tumor volume growth of the animal is shown in FIG. 11, which is used for evaluating the efficacy. The results show that compared with the vehicle control group, single administration of mDR18 (P<0.01) or combined administration of mDR18 and VSV (P<0.001) can significantly inhibit the tumor growth in the tumor-bearing mouse model of liver cancer. There is no statistically significant difference between the tumor inhibiting effects of single administration of mDR18 and combined administration of mDR18 and VSV, which indicates that combined administration of mDR18 and VSV has no synergistic effect.

### Example 4 Study on the Safety and Efficacy of Recombinant Oncolytic Vaccinia Virus Expressing mDR18 in Tumor-Bearing Immunocompetent Mouse Models of Melanoma

### 1. Experimental Materials

1.1 Oncolytic viruses: VV: it is a recombinant oncolytic vaccinia virus in which the TK gene is knocked out, and a genome structure of the oncolytic vaccinia virus VV (a recombinant oncolytic vaccinia virus in which the TK gene is knocked out) is shown in FIG. 5. VV-mDR18: a recombinant oncolytic vaccinia virus in which the TK gene is knocked out and that expresses the mouse DR18 gene, and a genome structure of the oncolytic vaccinia virus VV-mDR18 (a recombinant oncolytic vaccinia virus in which the TK gene is knocked out and that expresses the mouse DR18 gene) is shown in FIG. 6. The viruses were constructed by Guangzhou ViroTech Co., Ltd.

FIG. 6 shows the gene structure of the recombinant oncolytic vaccinia virus VV-mDR18 in which the TK gene is knocked out and that expresses the mouse DR18 gene.
1.2 Tumor cells: mouse melanoma cells B16-F10 were purchased from the China National Collection of Authenticated Cell Cultures.
1.3 Experimental animals: 5-7-week-old female C57BL/6J mice.
2.1 Construction of tumor-bearing mouse models

The tumor cells were thawed and cultured for passage. After the cells entered the logarithmic growth phase and the number of cells was sufficient, the cells were collected and counted, and a cell suspension was prepared. The B16-F10 cells were inoculated subcutaneously into the back of the C57BL/6J mice according to the inoculation quantity of 5×10⁵ cells/mouse.
2.2 Grouping and administration

After tumors were formed, the mice whose tumor volume was 100±40 mm³ were selected and randomly divided into 7 groups, namely, a vehicle control group, a low-dose VV group, a medium-dose VV group, a high-dose VV group, a low-dose VV-mDR18 group, a medium-dose VV-mDR18 group, and a high-dose VV-mDR18 group, 5 mice per group. Doses of the low/medium/high-dose VV or VV-mDR18 groups were respectively 1×10⁴¹¹ CCID50/mouse, the administration method was tail vein injection, and the administration was performed once. The day of tumor inoculation was denoted as DO, and the mice were observed for 14 days after administration.
2.3 Measurement of body weight and tumors of mice

During tumor formation and administration, the body weight and the tumor growth of the mouse were recorded every 3 or 4 days.
2.4 Data processing

The measured data was counted, and a tumor volume growth curve of the mice in each group was drawn.

A formula for calculating tumor volume (TV) is as follows: V=1/2×axb², where, a and b respectively represent a length and a width of a tumor.

Relative tumor volume (RTV)=Vₜ/V₀, where, V₀ is the tumor volume measured before grouping and administration, and Vₜ is the tumor volume at each measurement.

Relative tumor growth rate T/C (%)=T_{RTV}/C_{RTV}×100%, where, T_{RTV} represents RTV of the single administration or combined administration group of each drug, and C_{RTV} represents RTV of the vehicle control group.
2.5 Statistical analysis

The tumor volume and the body weight of the mouse were subjected to statistical analysis by repeated measures analysis of variance, * denotes P<0.05, ** denotes P<0.01, and *** denotes P<0.001, which all indicate that a difference between data is statistically significant.

### 3. Experimental Results

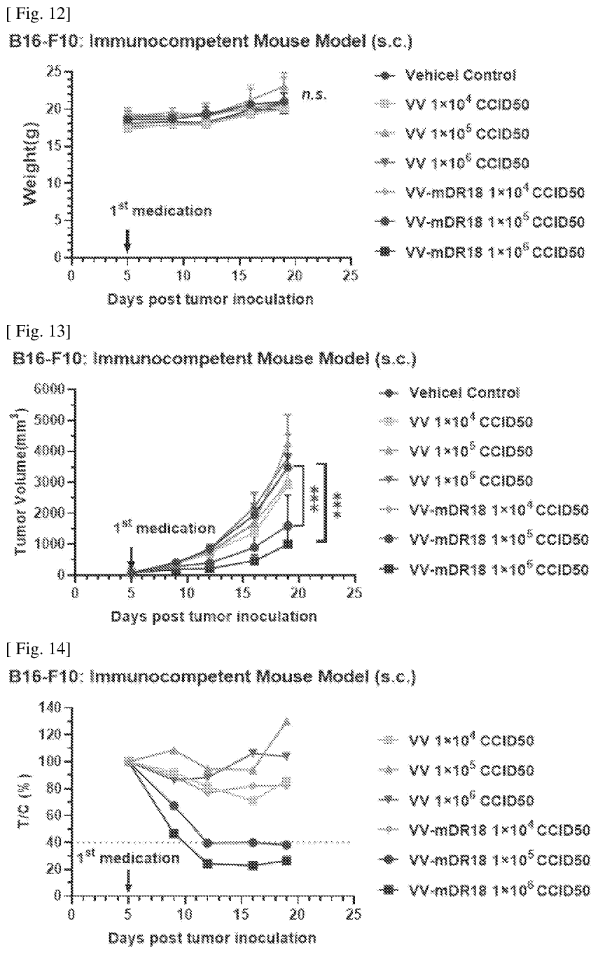

The body weight of the animal in each group during the experiment is shown in FIG. 12. The body weight of the animal in each group maintains a steady upward trend during the experiment. There is no obvious difference between the drug group of each dose and the vehicle control group. It indicates that tail vein injection of VV or VV-mDR18 does not cause an obvious drug-associated side effect in the immunocompetent mouse models.

A tumor volume change curve drawn based on the records of the tumor volume growth of the animal and a relative tumor growth rate curve are shown in FIG. 13 and FIG. 14, respectively, which are used for evaluating the efficacy. The results show that compared with the vehicle control group, the medium-dose group (1×10⁵ CCID50) and the high-dose group (1×10⁶ CCID50) of the recombinant oncolytic virus over-expressing mouse DR18 (VV-mDR18) can significantly inhibit the tumor growth (P<0.001). At the endpoint of the test, T/C values corresponding to the low-dose group (1×10⁴ CCID50), the medium-dose group (1×10⁵ CCID50), and the high-dose group (1×10⁶ CCID50) of VV-mDR18 are 82.07%, 38.08%, and 26.54%, respectively, which indicates that the tumor inhibiting effect of VV-mDR18 is dose-dependent.

### Example 5 Study on the Safety and Efficacy of Recombinant Oncolytic Vaccinia Virus Carrying mDR18 in Tumor-Bearing Immunocompetent Mouse Models

### 1. Experimental Materials

1.1 Oncolytic virus: VV-mDR18: it is a recombinant oncolytic vaccinia virus in which the TK gene is knocked out and that expresses the mouse DR18 gene, and a genome structure of the oncolytic vaccinia virus VV-mDR18 (a recombinant oncolytic vaccinia virus in which the TK gene is knocked out and that expresses the mouse DR18 gene) is shown in FIG. 6. The virus was constructed by Guangzhou ViroTech Co., Ltd.
1.2 Tumor cells: mouse bowel cancer cells MC-38, mouse lung cancer cells LLC, and mouse liver cancer cells H22 were purchased from Shenzhen Luoziman International Institute of Translational Medicine, China Center for Type Culture Collection, and Wuhan Procell Life Science & Technology Co., Ltd.
1.3 Experimental animals: 5-7-week-old female C57BL/6N mice and 5-7-week-old female Balb/c mice

### 2. Experimental Method

2.1 Construction of tumor-bearing mouse models

The tumor cells were thawed and cultured for passage. After the cells entered the logarithmic growth phase and the number of cells was sufficient, the cells were collected and counted, and a cell suspension was prepared. 1) The MC-38 cells were inoculated subcutaneously into the back of the C57BL/6N mice according to the inoculation quantity of 2.0×10⁶ cells/mouse. 2) The LLC cells were inoculated subcutaneously into the back of the C57BL/6N mice according to the inoculation quantity of 1.5×10⁶ cells/mouse. 3) The H22 cells were inoculated subcutaneously into the back of the Balb/c mice according to the inoculation quantity of 1.0×10⁶ cells/mouse.
2.2 Grouping and administration

After tumors were formed, the foregoing 3 kinds of tumor-bearing models were randomly divided into groups according to the tumor volume. 2 groups were set for each kind of model, which were a negative control group and a VV-mDR18 group, respectively. Each group included 5 mice. The day of tumor inoculation was denoted as DO. VV-mDR18 was administered by intratumor injection at a dose of 9.88×10⁵ PFU/mouse once a week for 6 weeks. Meanwhile, the same amount of normal saline was administered to the mice in the negative control group by intratumor injection.
2.3 Measurement of body weight and tumors of mice

During tumor formation and administration, the body weight and the tumor growth of the mouse were recorded every 3 or 4 days.
2.4 Data processing

The measured data was counted, and a tumor volume growth curve of the mice in each group was drawn.

A formula for calculating tumor volume (TV) is as follows: V=1/2×a×b², where, a and b respectively represent a length and a width of a tumor.

Relative tumor volume (RTV)=Vₜ/V₀, where, V₀ is the tumor volume measured before grouping and administration, and Vₜ is the tumor volume at each measurement.

Relative tumor growth rate T/C (%)=T_{RTV}/C_{RTV}×100%, where, T_{RTV} represents RTV of the single administration or combined administration group of each drug, and C_{RTV} represents RTV of the vehicle control group.
2.5 Statistical analysis

The tumor volume and the body weight of the mouse were subjected to statistical analysis by repeated measures analysis of variance, * denotes P<0.05, ** denotes P<0.01, and *** denotes P<0.001, which all indicate that a difference between data is statistically significant.

### 3. Experimental Results

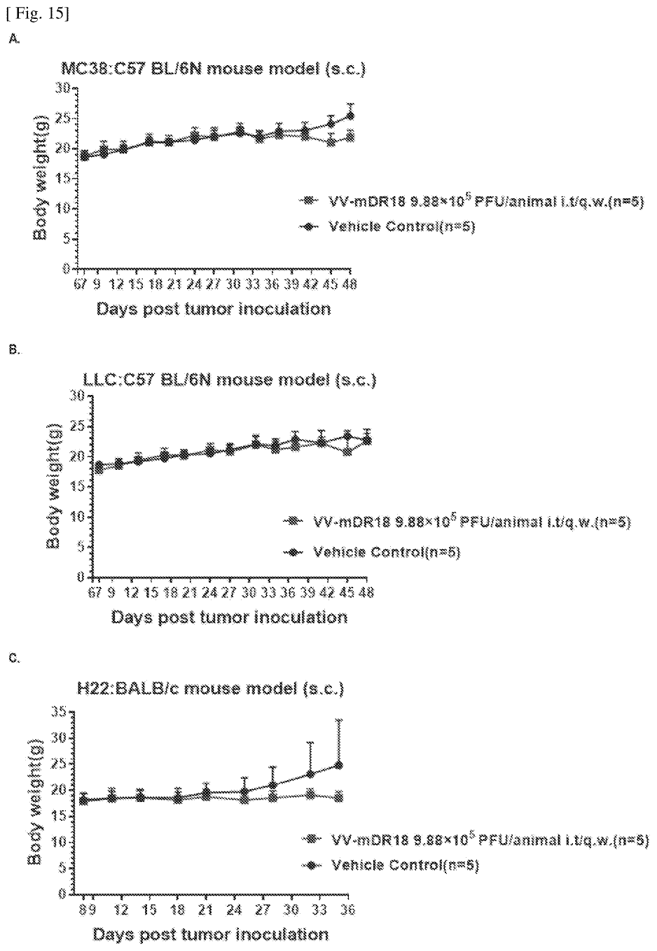

The body weight of the animal in each group during the experiment is shown in FIG. 15. The body weight of the animal in each group maintains a steady upward trend during the experiment. It indicates that intratumor injection of VV-mDR18 at the dose of 9.88×10⁵ PFU/mouse once a week does not cause an obvious drug-associated side effect in the immunocompetent mouse models.

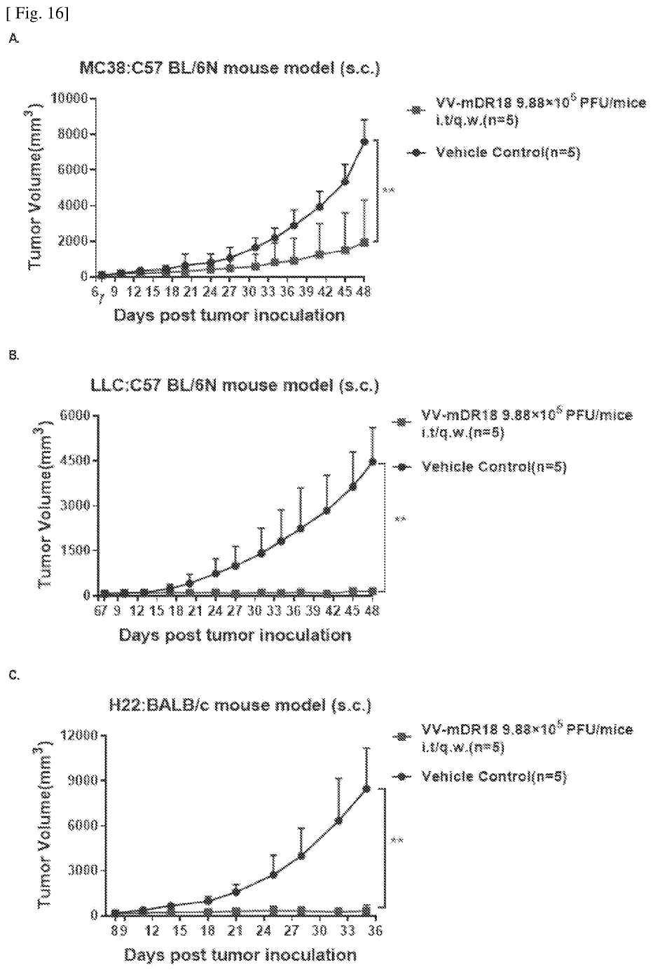

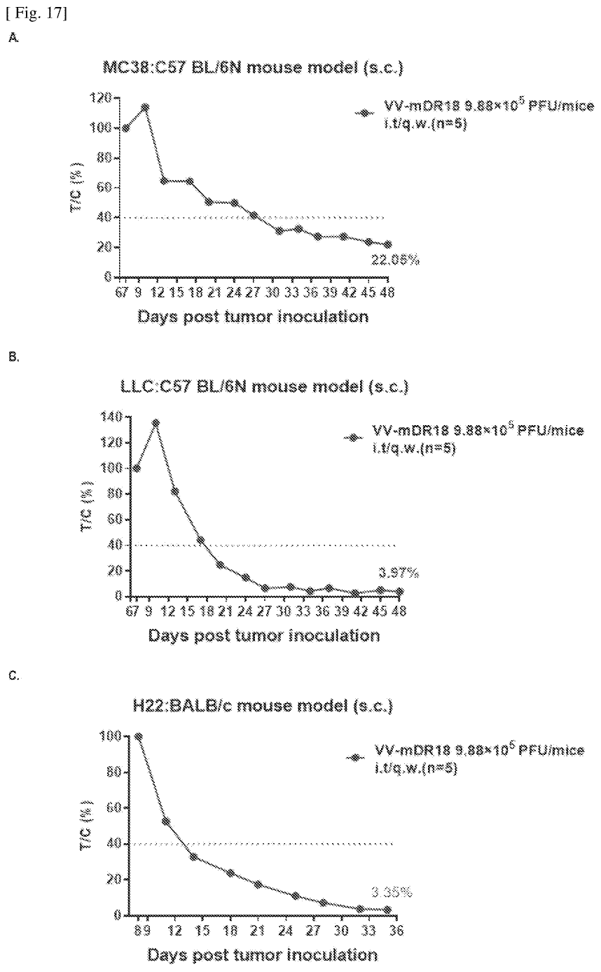

A tumor volume change curve drawn based on the records of the tumor volume growth of the animal and a relative tumor growth rate curve are shown in FIG. 16 and FIG. 17, respectively, which are used for evaluating the efficacy. The results show that compared with the negative control group, the recombinant oncolytic vaccinia virus carrying mouse DR18 (VV-mDR18) can significantly inhibit the tumor growth in the MC38 mouse models of bowel cancer, the LLC mouse models of lung cancer, and the H22 mouse models of liver cancer (P<0.01). At the endpoint of the test, T/C values corresponding to the 3 kinds of tumor-bearing models are 22.05%, 3.97%, and 3.35%, respectively.

An amino acid sequence of human DR-18 (SEQ ID
NO: 1)
(SEQ ID NO.: 1)
YFGKLESKLSVIRNLNDQVLFIDQGNRPLFEDMTDSDCRDNAPRTIFII
SKYSDSLARGLAVTISVKCEKISTLSCENKIISFKEMNPPDNIKDTKSD
IIFFQRDVPGHSRKMQFESSSYEGYFLACEKERDLFKLILKKEDELGDR
SIMFTVQNED
An amino acid sequence of murine DR-18 (SEQ ID
NO: 2)
(SEQ ID NO.: 2)
HFGRLHCTTAVIRNINDQVLFVDKRQPVFEDMTDIDQSASEPQTRLIIY
AYGDSRARGKAVTLSVKDSKMSTLSCKNKIISFEEMDPPENIDDIQSDL
IFFQKRVPGHNKMEFESSLYEGHFLACQKEDDAFKLILKKKDENGDKSV
MFTLTNLHQS
An amino acid sequence of wild-type human IL-18
(SEQ ID NO: 3)
(SEQ ID NO.: 3)
YFGKLESKLSVIRNLNDQVLFIDQGNRPLFEDMTDSDCRDNAPRTIFII
SMYKDSQPRGMAVTISVKCEKISTLSCENKIISFKEMNPPDNIKDTKSD
IIFFQRSVPGHDNKMQFESSSYEGYFLACEKERDLFKLILKKEDELGDR
SIMFTVQNED
An amino acid sequence of wild-type murine IL-18
(SEQ ID NO: 4)
(SEQ ID NO: 4)
NFGRLHCTTAVIRNINDQVLFVDKRQPVFEDMTDIDQSASEPQTRLIIY
MYKDSEVRGLAVTLSVKDSKMSTLSCKNKIISFEEMDPPENIDDIQSDL
IFFQKRVPGHNKMEFESSLYEGHFLACQKEDDAFKLILKKKDENGDKSV
MFTLTNLHQS
A nucleotide sequence of human DR-18 (SEQ ID
NO: 5)
(SEQ ID NO: 5)
TATTTCGGCAAGCTGGAAAGCAAGCTCTCCGTGATCAGAAACCTGAATG
ATCAGGTGCTGTTCATCGACCAGGGCAACCGGCCTCTGTTTGAGGACAT
GACCGACAGCGATTGCAGAGATAATGCCCCTAGAACCATCTTTATCATT
TCTAAGTACAGCGACAGCCTGGCTAGAGGCCTGGCCGTGACAATCAGCG
TGAAGTGCGAGAAGATCTCCACCCTGAGCTGCGAGAACAAGATCATCTC
TTTCAAGGAAATGAACCCCCCCGACAACATCAAGGACACAAAGTCCGAC
ATCATCTTCTTCCAGAGAGACGTGCCTGGCCACAGCAGGAAGATGCAGT
TCGAGAGCAGCAGCTACGAGGGCTACTTCCTGGCTTGTGAAAAAGAACG
GGACCTGTTTAAACTGATCCTGAAAAAGGAAGATGAGCTGGGAGATAGA
AGCATCATGTTCACCGTGCAGAACGAGGAC
A nucleotide sequence of murine DR-18 (SEQ ID
NO: 6)
(SEQ ID NO: 6)
CACTTTGGCCGACTTCACTGTACAACCGCAGTAATACGGAATATAAATG
ACCAAGTTCTCTTCGTTGACAAAAGACAGCCTGTGTTCGAGGATATGAC
TGATATTGATCAAAGTGCCAGTGAACCCCAGACCAGACTGATAATATAC
GCCTACGGCGACAGTAGAGCCAGAGGAAAGGCTGTGACCCTCTCTGTGA
AGGATAGTAAAATGTCTACCCTCTCCTGTAAGAACAAGATCATTTCCTT
TGAGGAAATGGATCCACCTGAAAATATTGATGATATACAAAGTGATCTC
ATATTCTTTCAGAAACGTGTTCCAGGACACAACAAGATGGAGTTTGAAT
CTTCACTGTATGAAGGACACTTTCTTGCTTGCCAAAAGGAAGATGATGC
TTTCAAACTCATTCTGAAAAAAAAGGATGAAAATGGGGATAAATCTGTA
ATGTTCACTCTCACTAACTTACATCAAAGT

Embodiments described in the present disclosure are illustrative examples only, and the embodiments of the present disclosure are not limited thereto. Any other change, modification, substitution, combination, and simplification made without departing from the spirit essence and principles of the present disclosure shall be deemed as equivalent replacement and shall fall within the scope of protection of the present disclosure.

1-15. (canceled)

16. A pharmaceutical combination comprising:
DR-18, and
an oncolytic vaccinia virus.

17. The pharmaceutical combination according to claim 16, wherein the pharmaceutical combination is a pharmaceutical composition or a medicine kit.

18. The pharmaceutical combination according to claim 17, wherein the pharmaceutical composition comprises the mixture of DR-18, and an oncolytic vaccinia virus.

19. The pharmaceutical combination according to claim 17, wherein the medicine kit comprising individually packaged DR-18 and an individually packaged oncolytic vaccinia virus.

20. The pharmaceutical combination according to claim 16, wherein the pharmaceutical combination is a recombinant oncolytic vaccinia virus with DR-18 nucleotide sequence inserted in the genome.

21. The pharmaceutical combination according to claim 16, wherein the DR-18 is human DR-18.

22. The pharmaceutical combination according to claim 21, wherein the human DR-18 comprises at least one mutation relative to wild-type human IL-18.

23. The pharmaceutical combination according to claim 21, wherein the human DR-18 comprises one or more of the following mutations relative to wild-type human IL-18 selected from the group consisting of M51K, K53S, Q56L, P57A, M60L, S105D, D110S, and N111R.

24. The pharmaceutical combination according to claim 21, wherein an amino acid sequence of the human DR-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 1.

25. The pharmaceutical combination according to claim 22, wherein an amino acid sequence of the wild-type human IL-18 comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence as shown in SEQ ID NO: 3.

26. The pharmaceutical combination according to claim 16, wherein the oncolytic vaccinia virus is a type with a defective TK gene.

27. The pharmaceutical combination according to claim 16, wherein the oncolytic vaccinia virus is selected from one or more of a WR strain, a Wyeth strain, a Lister strain, a Copenhagen strain, and a Tiantan strain.

28. The pharmaceutical combination according to claim 16, wherein a ratio of DR-18 to the oncolytic vaccinia virus is 0.01-200 mg: 10³-10⁹ PFU.

29. A recombinant oncolytic vaccinia virus, wherein a genome of the recombinant oncolytic vaccinia virus comprises:
a mutated oncolytic vaccinia virus nucleotide sequence, and
a DR-18 nucleotide sequence.

30. The recombinant oncolytic vaccinia virus according to claim 29, wherein the mutated oncolytic vaccinia virus nucleotide sequence comprises a functional defect of the TK gene.

31. The recombinant oncolytic vaccinia virus according to claim 29, wherein the DR-18 nucleotide sequence is a human DR-18 nucleotide sequence, and
the human DR-18 nucleotide sequence comprises a nucleotide sequence having at least 85%, at least 90%, at least 91%, at least 92%, at least 93%, at least 94%, at least 95%, at least 96%, at least 97%, at least 98%, at least 99%, or 100% identity to the nucleotide sequence as shown in SEQ ID NO:5.

32. The recombinant oncolytic vaccinia virus according to claim 31, wherein the oncolytic vaccinia virus is selected from one or more of a WR strain, a Wyeth strain, a Lister strain, a Copenhagen strain, and a Tiantan strain.

33. A method for the prevention and/or treatment of a tumor, comprising administering to a subject in need thereof the pharmaceutical combination according to claim 16.

34. The method according to claim 33, wherein the tumor is a solid tumor or blood tumor.

35. The method according to claim 34, wherein the solid tumor is selected from one or more of bowel cancer, pancreatic cancer, liver cancer, bladder cancer, breast cancer, cervical cancer, prostate cancer, glioma, melanoma, nasopharyngeal cancer, lung cancer, osteosarcoma, and gastric cancer.
